(19) United States
(12) Patent Application Publication
(10) Pub. No.: US 2022/0233605 A1
(43) Pub. Date: Jul. 28, 2022

(54) METHODS OF MAKING AND USING LIVER CELLS

(71) Applicant: University Health Network, Toronto (CA)

(72) Inventors: Shinichiro Ogawa, Toronto (CA); Mina Ogawa, Toronto (CA); Gordon Keller, Toronto (CA)

(21) Appl. No.: 17/616,522

(22) PCT Filed: Jun. 3, 2020
(86) PCT No.: PCT/IB2020/055249

§ 371 Date: Dec. 3, 2021

Related U.S. Application Data
(60) Provisional application No. 62/857,180, filed on Jun. 4, 2019.

(51) Int. Cl. A61K 35/407; C12N 5/071; A01N 1/02; G01N 33/50; A61K 35/413; A61P 1/16

(52) U.S. Cl. CPC A61K 35/407; G01N 2800/382; C12N 5/0671; A01N 1/0284; G01N 33/5041; G01N 33/5067; A61K 35/413; A61P 1/16; C12N 2513/00; C12N 2500/02; C12N 2506/45; C12N 2501/415; C12N 2501/15; C12N 2501/119; C12N 5/067

(57) ABSTRACT

Provided herein are methods of making and using a number of different types of liver cells.

### CROSS REFERENCE TO RELATED APPLICATIONS

This application claims the benefit of priority to U.S. Application No. 62/857,180 filed Jun. 4, 2019.

### TECHNICAL FIELD

This disclosure generally relates to stem cells and, more specifically, expansion and differentiation of stem cells.

### BACKGROUND

The liver is the largest solid organ and the largest gland in the human body. Classified as part of the digestive system, the liver carries out over 500 essential tasks including, for example, detoxification, protein synthesis, and the production of enzymes that help digest food. Despite the ability of the liver to regenerate, a diseased or malfunctioning liver can be dangerous or even fatal. Cell therapy is a viable alternative, but will require the ability to generate a number of different types of liver cells in large numbers. Additionally, the ability to generate a number of different type of liver cells allows for advancements in research.

### SUMMARY

This disclosure describes methods of making and using a number of different types of liver cells.

In one aspect, methods of expanding hepatoblasts are provided. Such methods typically include culturing the hepatoblasts in the presence of an activator of the Wnt pathway, a TGF beta inhibitor, and FGF19 or an equivalent thereof.

In some embodiments, the activator of the Wnt pathway is CHIR99021, CHIR98014, BIO, a (potent) GSK-3 inhibitor, or a natural Wnt agonists such as Wnt3. In some embodiments, the TGF-beta receptor inhibitor is SB431542, A83-01, or an ALK4 and/or ALK7 inhibitor (e.g., SB525334, SB505124, etc.). In some embodiments, the FGF19 or an equivalent thereof is an engineered version of FGF19 referred to as NGM282. In some embodiments, the method is performed under hypoxic conditions.

In another aspect, methods of expanding hepatoblasts are provided. Such methods typically include culturing the hepatoblasts under hypoxic conditions. In some embodiments, such methods further include culturing the hepatoblasts in the presence of an activator of the Wnt pathway, a TGF-beta receptor inhibitor, and FGF19 or an equivalent thereof.

In another aspect, methods of expanding hepatoblasts are provided. Such methods typically include culturing the hepatoblasts in the presence of an activator of the Wnt pathway, a TGF beta inhibitor, FGF19 or an equivalent thereof under hypoxic conditions. In some embodiments, within 3 to 5 passages, the number of hepatocytes are expanded about 100-fold to about 400-fold when cultured under ambient O2 conditions. In some embodiments, within 3 to 5 passages, the number of hepatocytes are expanded about 75-fold to about 1000-fold when cultured under hypoxic conditions. In some embodiments, an inhibitor of Notch signaling can be used in the culture to maintain the characteristics of hepatoblasts.

In another aspect, methods of obtaining mature hepatocytes are provided. Such methods typically include culturing hepatoblasts in the presence of a thyroid hormone or a thyroid hormone receptor agonist. In some embodiments, the thyroid hormone is triiodothyronine or thyroxine. In some embodiments, the thyroid hormone receptor agonist is GC-1. In some embodiments, the hepatoblasts are cultured as a monolayer. In some embodiments, the hepatoblasts are cultured as aggregates (plus thyroid hormone; also works in aggregates). In some embodiments, the hepatoblasts are cultured in the absence of cAMP. In some embodiments, the mature hepatocytes express little to no alpha fetal protein (AFP). In some embodiments, the mature hepatocytes express albumin. An inhibitor of Notch signaling can be used in the culture to maintain the characteristics of hepatocytes.

In some aspects, method of producing Zone1 hepatocytes are provided. Such methods typically include culturing hepatoblasts in the presence of an inhibitor of the Wnt pathway. In some embodiments, the inhibitor of the Wnt pathway is XAV939, IWP2, IWP4, or ICRT14. In some embodiments, the hepatoblasts are cultured in a monolayer or in aggregates.

In another aspect, methods of producing Zone3 hepatocytes are provided. Such methods typically include culturing hepatoblasts in the presence of an activator of the Wnt pathway. In some embodiments, the hepatoblasts are cultured in a monolayer or in aggregates.

In some aspects, methods of producing cholangiocytes are provided. Such methods typically include culturing hepatoblasts in the presence of retinoic acid, retinol or a RA receptor agonist. In some embodiments, cholangiocytes are identified based on the presence of a cystic fibrosis transmembrane conductance regulator (CFTR) protein. In some embodiments, cholangiocytes are identified based on binding to a DHC5-4D9 antibody.

In another aspect, methods of producing liver organoids are provided. Such methods typically include combining mesothelial cells (US20160215263) with hepatoblasts under conditions that promote self-assembly into liver organoids. In some embodiments, such methods further include expanding the hepatoblasts in the presence of an activator of the Wnt pathway, a TGF beta inhibitor, and FGF19.

In another aspect, methods of producing stellate cells are produced. Such methods typically include culturing the liver organoids as described herein under conditions in which stellate cells are produced.

In another aspect, methods of treating liver disease in a subject (e.g., a cholangiopathy such as, without limitation, a bile duct disease or a paucity) are provided. Such methods typically include transplanting a composition comprising cholangiocytes into the subject.

In another aspect, methods of treating a subject having liver disease are provided. Such methods typically include transplanting a composition comprising hepatoblasts expanded using any of the methods described herein; transplanting a composition comprising hepatocytes matured using any of the methods described herein; transplanting a composition comprising zone 1 hepatocytes made using any of the methods described herein; transplanting a composition comprising zone 3 hepatocytes made using any of the methods described herein; transplanting a composition comprising cholangiocytes made using any of the methods described herein; transplanting a composition comprising the liver organoids as described herein into the subject; and/or transplanting a composition comprising stellate cells made using any of the methods described herein. In some embodiments, the composition further comprises epithelial cells. In some embodiments, the methods further include monitoring the subject for albumin levels. In some embodiments, the methods further include monitoring the subject for the level of one or more liver enzymes (total bilirubin, aspartate transaminase (AST), alanine transaminase (ALT), or gamma-glutamyl transferase (GTP)). In some embodiments, the transplanting is directly into the liver or ectopic in the abdomen.

In another aspect, methods of culturing liver cells are provided. Such methods typically include culturing liver cells on a substrate under conditions in which the liver cells grow as a monolayer. In some embodiments, the methods further include culturing the liver cells as aggregates following their culturing as a monolayer. In some embodiments, the number of liver cells resulting from the monolayer is at least 10-fold (e.g., 15-fold, 20-fold) greater than the number of liver cells resulting from a culture of aggregate cells.

In still another aspect, methods of cryopreserving liver cells are provided. Such methods typically include culturing the liver cells in the presence of an activator of the Wnt pathway, a TGF beta inhibitor, and FGF19 or an equivalent thereof for at least 3 days; and cryopreserving the cultured liver cells. Such methods further can include thawing the cryopreserved liver cells and culturing the thawed liver cells in the presence of an activator of the Wnt pathway, a TGF beta inhibitor, and FGF19 or an equivalent thereof.

In yet another aspect, methods of recovering cryopreserved liver cells are provided. Such methods typically include thawing the cryopreserved liver cells; and culturing the thawed liver cells in the presence of an activator of the Wnt pathway, a TGF beta inhibitor, and FGF19 or an equivalent thereof. Such methods further can include culturing the liver cells in the presence of an activator of the Wnt pathway, a TGF beta inhibitor, and FGF19 or an equivalent thereof for at least 3 days prior to cryopreserving the liver cells.

In some embodiments, cryopreservation comprises freezing the liver cells at −80° C. in media comprising DMSO, FSC and DMEM/F12. In some embodiments, thawing comprises heating the liver cells to 37° C. for about 5 mins. In some embodiments, the liver cells are hepatoblasts.

In another aspect, methods of screening for compounds therapeutic for cystic fibrosis and/or ciliopathy are provided. Such methods typically include contacting cholangiocytes with a test compound, and determining the presence or absence of CFTR function. Generally, the presence or absence of CFTR function is indicative of a test compound that is therapeutic for cystic fibrosis and/or ciliopathy.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing the different cell types in the adult liver.

FIG. 2A shows expansion of the hPSC-derived hepatoblast population.

FIG. 2B shows that hepatoblasts within the expanded population retained their capacity to differentiate, cells from the third passage were differentiated along both the hepatic and cholangiocyte fates.

FIG. 3A is a schematic showing that a hepatoblast can be cultured under hypoxia condition in the presence of 3 pathway modulators, and can be serially expanded up to a total of 10 passages.

FIG. 3B is a graph showing fold expansion when a hepatoblast is cultured under hypoxia condition in the presence of 3 pathway modulators.

FIG. 3C is a plot showing the distribution of AFP+ ALB+ expressing cells produced under ambient O2 conditions or hypoxia O2 conditions.

FIG. 4 shows that thyroid hormone (T3) promotes maturation of hPSC-derived hepatocytes.

FIG. 5 shows that the Wnt signaling pathway regulates zonation of hPSC-derived hepatocytes.

FIG. 6A is a schematic showing the differentiation of progenitor cells into zone 1- and zone 3-like cells.

FIG. 6B shows that hepatoblasts cultured under monolayer conditions expressed albumin and repressed AFP in a monolayer.

FIG. 6C is a graph of qPCR analysis.

FIG. 6D is a schematic showing the estimated number of differentiated Zone 1- and Zone 3-like hepatocytes from one ES cells following the expansion protocol described herein.

FIG. 7 shows that retinoic acid signaling promotes the generation of CFTR expressing cholangiocytes (bile duct cells) in monolayer cultures.

FIG. 8A-8D shows that identification of signaling pathways that promote the development of ciliated cholangiocytes, which are functional bile duct cells, in monolayer cultures.

FIG. 9 shows the characterization of NFR-induced cholangiocytes.

FIG. 10 shows that hPSC-derived mesothelial cells support hepatoblast function in vitro.

FIG. 11 shows engraftment of hPSC-derived cholangiocytes.

FIG. 12 shows the subcutaneous (ectopic) transplantation and engraftment of hepatic organoids.

FIG. 13 shows the intra-abdominal (ectopic) transplantation and engraftment of hepatic organoids.

FIG. 14A shows representative flow cytometry of ALB and AFP expression in the hepatoblast population following 8 days of culturing the thawed cryopreserved cells.

FIG. 14B shows the percent of ALB and AFP positive cells in the expanded hepatoblast population following 8 days of culturing the thawed cryopreserved cells (“−”, cryopreserved without expansion; “+”, cryopreserved expanded population).

FIG. 14C is a graph showing fold-expansion of the hepatoblast population following 8 days of culturing the cryopreserved cells. Values are compared to the number of cells plated immediately following the thaw (“−”, cryopreserved without expansion; “+”, cryopreserved expanded population).

FIG. 14D shows representative flow cytometric analyses of ALB and AFP expression in Zone 1 and Zone 3 hepatocytes generated from cryopreserved hepatoblasts.

FIG. 15A shows the scheme and timelines for hepatoblast expansion and zone maturation prior to the ectopic transplantation of kidney subcapsule in NSG mice.

FIG. 15B is a graph showing the levels of human albumin in the sera of mice 4 weeks following engraftment of the indicated populations. Aggregates of the indicated populations were grafted to the kidney capsule of NSG mice. Zone1/Zone3: equal numbers of Zone 1 and Zone 3 aggregates were mixed and engrafted. Data are represented as mean+/−SEM, * indicates P<0.05, *** indicates P<0.0001, statistical analysis: one-way ANOVA.

### DETAILED DESCRIPTION

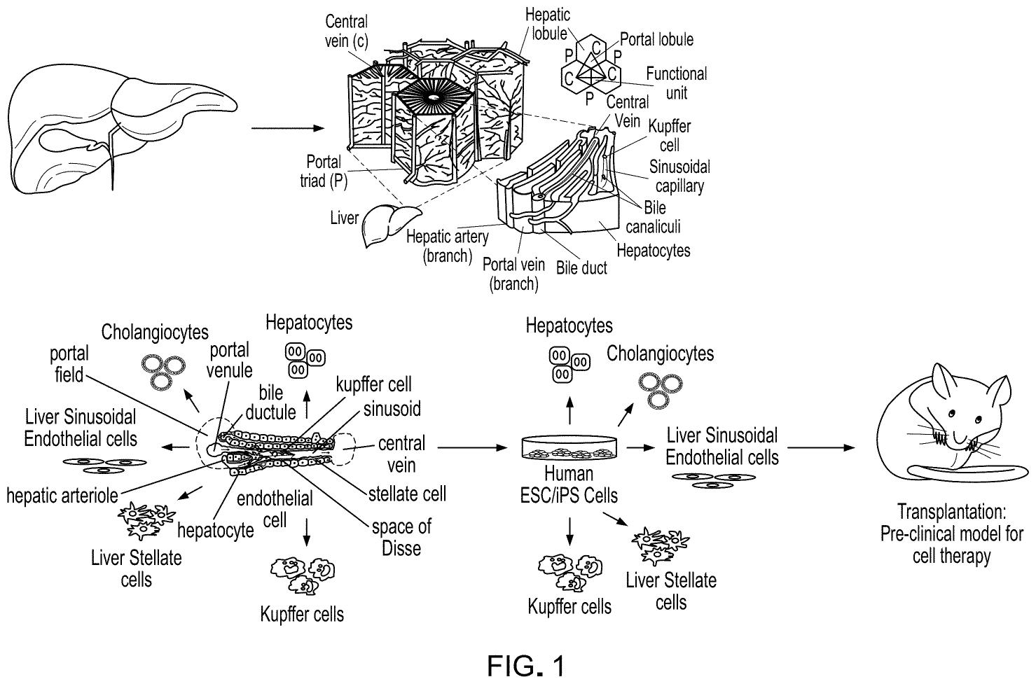

The adult liver is a complex tissue that contains multiple cell types of both endodermal and mesodermal origin including hepatocytes, cholangiocytes, liver sinusoidal endothelial cells, liver stellate cells and Kupffer cells. FIG. 1 is a schematic showing the different cell types in the adult liver. To be able to generate functional liver tissues derived from human pluripotent stem cells (hPSC) in vitro or in vivo, it likely will be necessary to include most, if not all of these cell types, in the engineered structure. This disclosure describes methods of making a number of the liver cells shown in FIG. 1, and also describes a number of ways in which such liver cells can be used.

### Hepatocytes and Cholangiocytes

Hepatocytes make up the parenchyma of the liver and represent approximately 75% of the total cell population in the organ. These cells perform over 3,000 essential functions within the body that involve different enzyme reactions occurring at the same time. To achieve this, hepatocytes with different functions are compartmentalized into different zones of the parenchyma. Recent single cell RNA-SEQ studies have shown that approximately half of the genes expressed in mouse hepatocytes are zonated. The region surrounding the portal vein is known as Zone1 and the hepatocytes in this region (“Zone1 hepatocytes”) mainly contribute to gluconeogenesis and urea synthesis. In contrast, the region around the central vein is known as Zone3 and the hepatocytes in this region (“Zone3 hepatocytes”) are responsible for xenobiotic metabolism.

As described herein, the strategy to generate functional hepatic cells from hPSCs involves specific steps that recapitulate the critical stages of liver development in the early embryo, including the induction of the proper hepatic progenitor cells (hepatoblasts) and maturation to a hepatocyte with zonal functional heterogeneity. Using this approach, new insights into hepatic development from hPSCs have been identified, enabling the derivation of cells that display the distinct characteristics of primary human hepatocytes with zonal distribution. With these advances, it is possible to generate hPSCs-derived populations that comprise the functional heterogeneity of primary hepatocytes that make up the portal vein to central vein axis of the liver.

In addition to hepatocytes, cholangiocytes also play an important role in liver function, as they form the bile ducts that carry bile acid. Additionally, cholangiocytes also modify the bile acid as it flows through the duct. Although the cholangiocytes represent only 5% of total liver mass, they are directly related to a number of different diseases that can lead to liver failure. Liver disease related to biliary failure accounts for 80% of pediatric liver transplantation. Over the past decade, a number of groups have invested significant time and effort into generating hepatocyte-like and cholangiocyte-like cells from human pluripotent stem cells (hPSCs). Despite this, the generation of functional mature zonated hepatocytes and mature ciliated cholangiocytes has not been previously achieved.

As described herein, RA signaling was identified as a regulator of early cholangiocyte specification and the combination of BMP inhibition, Rho-kinase and cAMP signaling in the maturation of hPSCs-derived cholangiocytes. The staged manipulation of these pathways promotes efficient development of functional CFTR-positive ciliated cholangiocytes from hPSCs. In addition, cholangiocytes in monolayer efficiently generate cholangiocyte cysts and organoids.

This disclosure describes methods of generating functional hepatocytes and functional cholangiocytes, which can take place in either a monolayer format or an aggregation/organoid format. This disclosure also describes methods to expand the heptatoblast population under conditions that maintain their ability to differentiate and generate functional hepatocytes and cholangiocytes, which can take place in either a monolayer or an aggregation/organoid format. It would be understood that any of the liver cells described herein can be grown as aggregates prior to and/or after those liver cells are grown in a monolayer. When cells (e.g., hepatocytes) are grown in a monolayer, inhibition of both Notch and TGF-beta signaling can improve the quality of maturation (e.g., relative to the maturation of the same type of cells grown in a 3D culture). As described herein, the number of liver cells that can be obtained from culturing in a monolayer can be at least 10-fold (e.g., at least 15-fold, at least 20-fold) greater than the number of liver cells that can be obtained from culturing in aggregates. Further, the methods described herein result in a significant reduction of AFP-positive cells under the hepatic maturation condition together with the manipulation of Wnt signaling for hepatic zonation compared to previously published methods (Ogawa et al., 2013, Development, 140(15):3285-96).

### Methods of Expanding Liver Progenitor Cells

A number of different methods to expand hepatoblasts are described herein. In some embodiments, hepatoblasts can be significantly expanded in number by culturing the cells in the presence of a cell expansion cocktail. As described herein, a cell expansion cocktail typically includes an activator of the Wnt pathway, a TGF beta inhibitor, and FGF19 or an equivalent thereof. These culture conditions enable serial expansion of hepatoblasts, with a 6- to 8-fold increase in cell number at each expansion. The hepatoblast population can be serially expanded for at least 10 passages while maintaining the functional characteristics of hepatic progenitor cells (e.g., over 90% of the cells express both ALB and AFP).

Activators of the Wnt pathway are known or can be identified by a skilled artisan. Representative activators of the Wnt pathway include, without limitation, CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile; TOCRIS), CHIR98014 (N6-[2-[[4-(2,4-Dichlorophenyl)-5-(1H-imidazol-1-yl)-2-pyrimidinyl]amino]ethyl]-3-nitro-2,6-pyridinediamine; TOCRIS), BIO ((2′Z,3′E)-6-Bromoindirubin-3′-oxime; TOCRIS), any number of (potent) GSK-3 beta inhibitors, or natural Wnt agonists such as Wnt3. Inhibitors of the TGF-beta receptor are known or can be identified by a skilled artisan. Representative inhibitors of the TGF-beta receptor include, without limitation, SB431542, A83-01, other TGF beta receptor inhibitors, or an ALK4 and/or ALK7 inhibitor (e.g., SB525334, SB505124, etc.). FGF19 is known in the art. See, for example, GI Accession No. 37181724 for the protein sequence of the human FGF19. In addition, equivalents of FGF19 are known and include, for example, an engineered version referred to as NGM282.

Additionally or alternatively, hepatoblasts can be expanded significantly in number by culturing the cells under hypoxic conditions. Hypoxic conditions are known in the art. With respect to cell culture, ambient oxygen (O2) conditions generally refer to a level of oxygen in the culture of about 20% O2 (e.g., about 18%, 20%, 22.5% or 25% O2), while hypoxic conditions generally refer to a level of oxygen in the culture of less than about 20% O2 (e.g., about 15%, 10%, 5%, or 2.5% O2).

As described herein, hepatoblasts can be expanded to very high numbers by culturing the cells in the presence of a cell expansion cocktail under hypoxic conditions. Based on preliminary results presented herein, it is predicted that, within 3 to 5 passages, hepatocytes can be expanded about 100-fold to about 400-fold when cultured in the presence of a cell expansion cocktail under ambient O2 conditions and about 75-fold to about 1000-fold when cultured in the presence of a cell expansion cocktail under hypoxic conditions.

### Methods of Generating Liver Cells

Methods of making a number of different types of liver cells also are described herein. For example, methods of making mature hepatocytes are described, including Zone 1-like hepatocytes and Zone 3-like hepatocytes, and methods of making cholangiocytes also are described.

In some embodiments, mature hepatocytes can be obtained by culturing hepatoblasts in the presence of a thyroid hormone or a thyroid hormone receptor agonist. Mature hepatocytes generally are characterized as hepatocytes that express albumin and express little to no (detectable) alpha fetal protein (AFP). Thyroid hormones are known in the art, as are thyroid hormone receptor agonists. Representative thyroid hormones include, without limitation, triiodothyronine or thyroxine, while a representative thyroid hormone receptor agonist is GC-1. Notably, an increased number of mature hepatocytes can be obtained by culturing hepatoblasts in the presence of little to no cAMP.

As described herein, the zonation of hepatoblasts can be facilitated by manipulating Wnt signaling in the cells together with the treatment of thyroid hormone. Zone 1 hepatocytes (or Zone 1-like hepatocytes) can be obtained by culturing hepatoblasts in the presence of an inhibitor of the Wnt pathway. Inhibitors of the Wnt pathway are known or can be identified by a skilled artisan; representative Wnt pathway inhibitors include, without limitation, XAV939, IWP2, IWP4, or ICRT14 (see, for example, selleckchem.com/Wnt on the World Wide Web). Zone 3 hepatocytes (or Zone 3-like hepatocytes) can be obtained by culturing hepatoblasts in the presence of an activator of the Wnt pathway. Activators of the Wnt pathway are discussed herein and include, without limitation, CHIR99021, CHIR98014, BIO, GSK-3 inhibitors and natural Wnt agonists (e.g., Wnt3). Zone3 hepatocytes express multiple CYP enzymes including, without limitation, CYP2C9, CYP2D6 and CYP3A4, which are highly expressed in pericentral hepatocytes in the liver lobule, whereas Zone1 hepatocytes express PCK, G6P, TAT and CPS1, which are highly expressed in periportal hepatocytes (Zone1) in the liver lobule.

In some embodiments, cholangiocytes can be obtained by culturing hepatoblasts in the presence of retinoic acid, retinol or a RA receptor agonist. It would be appreciated that cholangiocytes can be identified based on the expression of the cystic fibrosis transmembrane conductance regulator (CFTR) protein, and also can be identified based on binding to a DHC5-4D9 antibody (Millipore Sigma: MABS2040-100 μg; Anti-Hpd3 antibody, clone DHIC-4D9).

In some embodiments, liver organoids can be obtained by combining mesothelial-like cells with hepatoblasts under conditions that promote self-assembly into liver organoids. Suitable mesothelial-like cells can be generated, for example, by following the protocol that is used to produce cardiac epicardial cells in US 2016/0215263. In some embodiments, hepatic stellate-like cells can be obtained from the liver organoids (e.g., by culturing the liver organoids described herein under conditions in which hepatic stellate-like cells are spontaneously produced in 3D liver organoids in the presence of a Wnt agonist, a TGF beta inhibitor, and FGF19 or an equivalent thereof, and subsequently maintained under the hepatic maturation conditions with the manipulation of hepatic zonation).

### Cryopreservation

Liver cells such as those described herein (e.g., hepatoblasts) can be cryopreserved, and the expansion cocktail (i.e., an activator of the Wnt pathway, a TGF beta inhibitor, and FGF19 or an equivalent thereof) and conditions for expansion as described herein can be used after cryopreservation and subsequent thawing to allow for improved recovery and maintenance of the cells. Use of the expansion cocktail and associated conditions described herein following cryopreservation can result in greater than 85% of the cells being viable following thawing, and, significantly, those cells generally maintain the characteristics of the hepatic progenitor cells.

Prior to freezing the liver cells, such cells can be cultured in the expansion cocktail under the expansion conditions described herein. Culturing in the expansion cocktail prior to freezing of the cells also can be used to improve the ability of the cells to recover and expand following cryopreservation of the cells.

As used herein, cryopreservation refers to freezing the cells (e.g., at −80 C) in media that includes DMSO, FSC and DMEM/F12. Thawing, on the other hand, can be done by gently heating the cells (e.g., at 37° C.) for about 5 minutes.

### Therapeutic Methods

Any of the liver cells described herein (e.g., expanded hepatoblasts, mature hepatocytes, Zone 1 hepatocytes, Zone 3 hepatocytes, cholangiocytes, liver organoids, stellate cells, and combinations thereof) can be used therapeutically to treat a number of different liver diseases. It would be understood that, in the context of cell therapy, administration generally refers to the introduction (e.g., via transplantation) of cells into a subject. In the case of introducing liver cells into a subject, transplantation can be directly into the liver or ectopic to the liver (e.g., in the abdomen).

In some embodiments, for example, a composition that includes cholangiocytes made using the methods described herein can be transplanted into a subject having a liver disease (e.g., a cholangiopathy such as, for example, a bile duct disease or a paucity). It would be understood that, in addition to introducing any of the liver cells described herein into a subject, non-liver cells also can be introduced into the subject as a part of the transplantation. A non-limiting example of non-liver cells includes, for example, epithelial cells.

As used herein, subjects generally refers to humans, but also could refer to any other type of animal (e.g., mammals or non-mammals; e.g., companion animals, farm animals or livestock, exotic animals). Following transplantation, the subject often is monitored for a product or by-product of the transplanted cells in order to determine the health and functionality of the transplanted cells. For example, subjects receiving liver cells can be monitored for albumin levels and/or the level of one or more liver enzymes (e.g., total bilirubin, aspartate transaminase (AST), alanine transaminase (ALT), gamma-glutamyl transferase (GTP), or combinations thereof).

As described herein, mature cholangiocytes, produced in either monolayer or 3D culture format, are able to engraft both intrahepatic and extrahepatic sites to form ductal-like structures, providing a platform for the development of novel therapeutic applications for the treatment of biliary cholestatic diseases. A skilled artisan would understand that “treating” or “treatment” typically refers to reducing, ameliorating or mitigating a disease, the effects of the disease, or one or more symptoms associated with the disease.

### Drug Screening and Laboratory Methods

Any of the liver cells described herein can be used in drug screening protocols. For example, the cholangiocytes described herein can be used to screen for compounds that may exhibit therapeutic benefits in the treatment of cystic fibrosis and/or ciliopathy. For example, such methods typically include contacting liver cells with a test compound, and determining the presence or absence or amount of one or more “markers”. As used herein, a “marker” can refer to a particular functionality of a protein or of the cell, or a “marker” can refer to the expression of a particular sequence. For example, such methods typically include contacting cholangiocytes with a test compound, and determining the presence or absence of CFTR function (e.g., chloride channel function). It would be understood that the presence or absence of CFTR function is indicative of a test compound that may exhibit therapeutic benefits in the treatment of cystic fibrosis and/or ciliopathy.

The cells described herein can be evaluated using, for example, a FLIPR assay (fluorescent based plate reader assay) and membrane potential dye can be used to measure apical chloride conductance, which is indicative of CFTR function. The cells described herein can be evaluated for a Z prime score to determine the quality control; as determined herein, the Z prime score for the cholangiocytes described herein was 0.63, indicating that such cells are excellent candidates for CFTR drug screening.

Since the mature cholangiocytes described herein (i.e., produced in either monolayer or 3D culture format) are functional, they can be used, for example, in high-throughput drug screening assays to measure, for example, CFTR function. Such cells also can be used, for example, in assays to examine or determine chemo-sensing and/or mechano-sensing activity (based on the movement of the primary cilia).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1A

### Experimental Materials and Methods for Expanding Liver Progenitor Cells

### Expansion of Hepatoblasts

Day 27 hepatoblasts were dissociated with TrypLE (Thermo Fisher Scientific) as a single cell and plated on 2.5% Matrigel coated well (12 well plates) at a concentration of 200,000 cell per well in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), CHIR99021 (1 μM), SB431542 (6 μM) and FGF19 (50 ng/ml). The medium was changed every two days or three days. The cell culture could be maintained in either an ambient incubator (5% CO2, 20% O2, 90% N2 environment) or a low O2 incubator (5% CO2, 5% O2, 90% N2 environment). The plated hepatoblasts were proliferated and became fully confluent within 6-10 days. The proliferated hepatoblasts were also able to expand by another passage with a single cell dissociation by TrypLE (Thermo Fisher Scientific). Compared to the culture in an ambient O2 incubator, the expanded hepatoblasts in a low O2 incubator are able to further expand up to 10 times passage with an appearance of over 90% of both ALB and AFP positivity. The expanded hepatoblasts were also able to differentiate into zone 1/3 hepatocyte-like cells and cholangiocytes following the monolayer protocol described above.

### Example 1B

### Experimental Results for Expansion of Liver Progenitor Cells

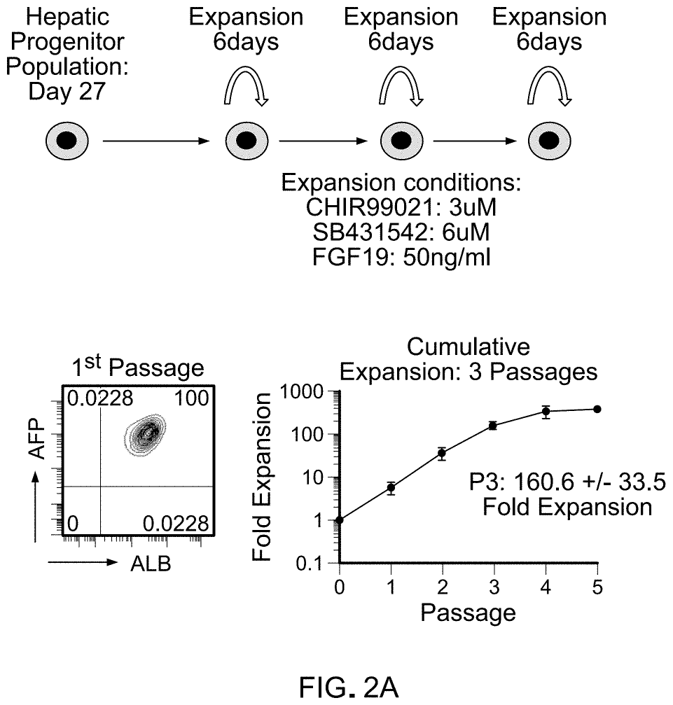

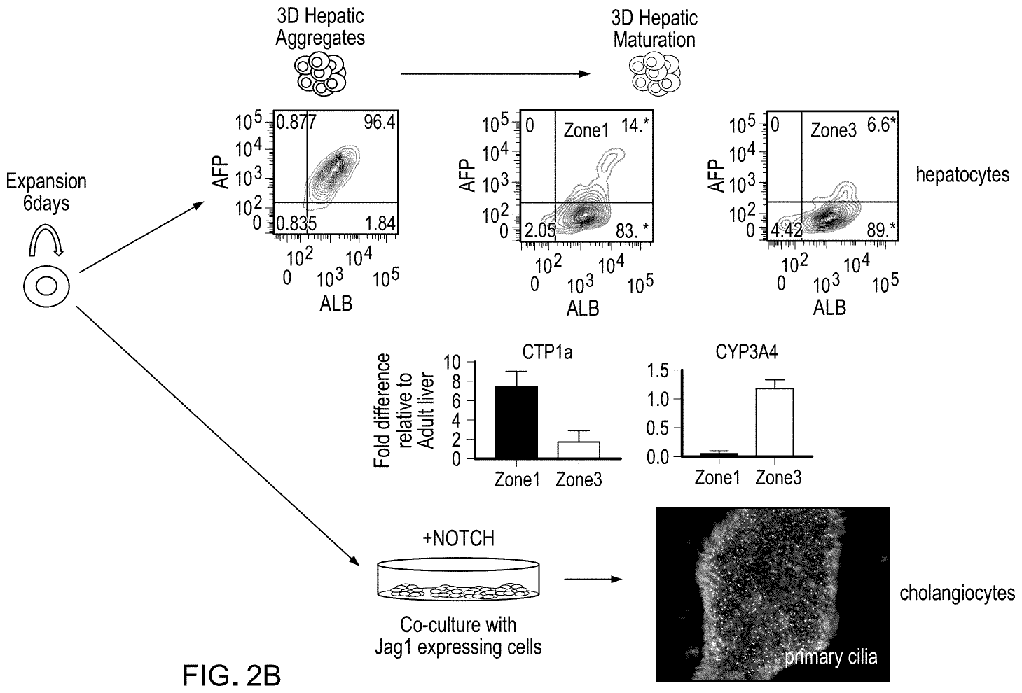

FIG. 2A shows expansion of the hPSC-derived hepatoblast population. To be able to generate sufficient numbers of hPSC-derived hepatic cells for cell-based therapy, it would be advantageous to be able to expand and cryopreserve the bi-potential hepatoblast population. To achieve this, hepatoblasts were cultured in the combination of a Wnt signaling agonist (CHIR), a TGF beta signaling antagonist (SB431542), and FGF19 or an equivalent thereof. The activation/inhibition of these pathways plays a role in liver regeneration and promotes the proliferation of hepatocytes in the normal liver and pre-cancerous liver. When cultured under these conditions, the hepatoblasts proliferate and maintain their ALB+ AFP+ profile. Cells can be passaged every 6 days for a total of 3 passages, resulting in a total expansion of 160-fold; cells appear to lose their proliferative potential beyond passage 3.

FIG. 2B shows that hepatoblasts within the expanded population retained their capacity to differentiate, cells from the third passage were differentiated along both the hepatic and cholangiocyte fates. The cells within the expanded population generated Zone 1- and Zone 3-like ALB+ AFP− hepatocytes that expressed PCK1 and CPT1a (Zone 1-like cells) or CYP3A4 and CYP2D6 (Zone 3-like cells) following culturing in the presence of T3 and a Wnt agonist or antagonist. Additionally, cells in the expanded population from the first, second and third passages also differentiated along the cholangiocyte lineage and gave rise to ciliated cells.

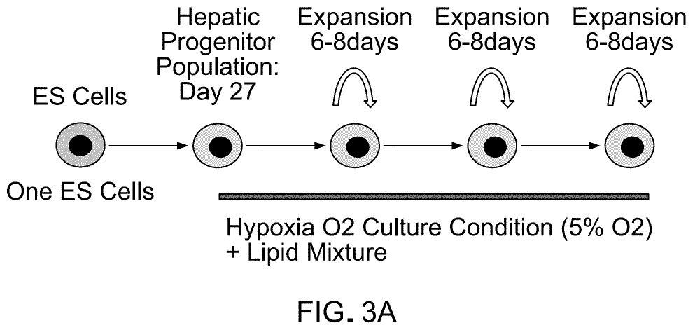

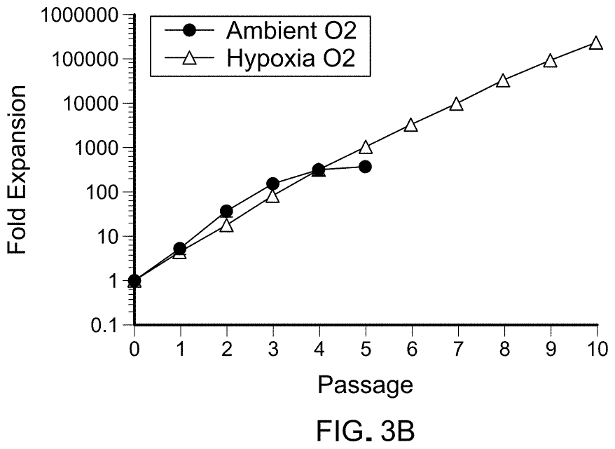

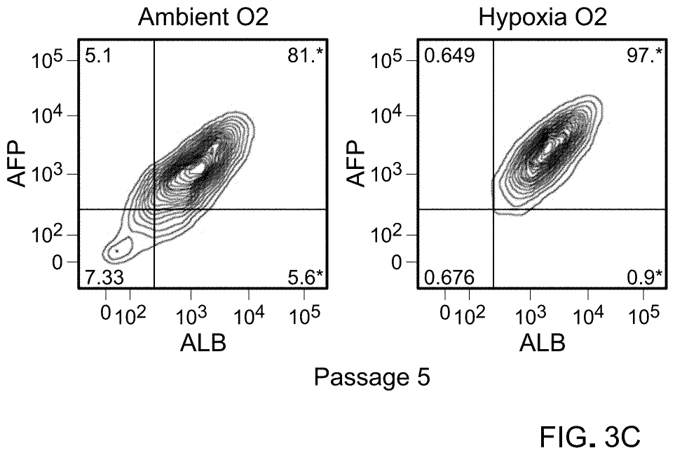

FIG. 3A is a schematic showing that a hepatoblast can be cultured under hypoxia condition in the presence of 3 pathway modulators, and can be serially expanded up to a total of 10 passages.

FIG. 3B is a graph showing fold expansion when a hepatoblast is cultured under hypoxia condition in the presence of 3 pathway modulators. For example, at passage 5, there was a 388-fold expansion in ambient O2 and a 1076-fold expansion in hypoxia O2 condition. A 237404-fold expansion is projected after 10 passages.

FIG. 3C is a plot showing the distribution of AFP+ ALB+ expressing cells produced under ambient O2 conditions or hypoxia O2 conditions.

### Example 2A

### Experimental Materials and Methods for Generating Liver Cells

### Hepatocytes and Zonation of Hepatocytes

Human ES and iPS Cells Maintenance and Differentiation into Hepatoblast

Human ES/iPS cells were maintained on irradiated mouse embryonic feeder cells in human ES culture medium consisting of DMEM/F12 (50:50: Gibco) supplemented with 20% knock-out serum replacement as described previously. Prior to the induction of endoderm in the monolayer culture, hES/iPS cells were passaged onto a 2.5% Matrigel coated surface (10-fold less than previous protocols) for 1 day at the cell density of 200,000 cell per well in a 12 well culture dish. To induce endoderm differentiation, the cells were cultured for 1 day in RPMI based medium supplemented with glutamine (2 mM), MTG (4.5×10E-4 M; Sigma), activin A (100 ng/ml), and CHIR99021 (2 μM). At dayl, CHIR99021 was removed and cells were cultured for the next 2 days in RMPI supplemented with glutamine (2 mM), ascorbic acid (50 μg/ml:Sigma), MTG (4.5×10E-4M; Sigma), basic fibroblast growth factor (bFGF, 5 ng/ml), activin A (100 ng/ml) followed by 4 days in serum-free-differentiation (SFD) based medium with the same supplements. Then media was changed every two days. At day 7, the definitive endoderm, which is confirmed as positive for CXCR4 and cKIT by flow cytometry, was specified to a hepatic fate by culture in H16 DMEM containing bFGF (40 ng/ml) and Bone Morphogenic Protein (BMP4, 50 ng/ml) and supplemented with 1% vol/vol B27 supplement (Invitrogen, A11576SA), ascorbic acid, and MTG. The media was changed every 2 days from day 7 to day 13. To promote the maturation of the hepatoblast population, cells were cultured in a mixture of H16 DMEM/Ham's F12 (3:1) media with 0.1% BSA, 1% vol/vol B27 supplement, ascorbic acid, glutamine, MTG, Hepatocyte Growth Factor (HGF, 20 ng/ml), Dexamethasone (Dex, 40 ng/ml) and Oncostatin M (OSM; 20 ng/ml), CHIR99021 (1 μM) for 8 days. The differentiation including the endoderm induction, hepatic specification and maturation from day 0 to day 21 were maintained in a low O2 incubator in a 5% CO2, 5% O2, 90% N2 environment. At day 21, the cells were transferred into an ambient 02 incubator and cultured in a mixture of H21 DMEM/Ham's F12 (3:1) with 0.1% BSA, 1% vol/vol B27 supplement, ascorbic acid, glutamine, MTG, HGF (20 ng/ml), Dex (40 ng/ml) and OSM (20 ng/ml) for 4 days. At day 25, cells were cultured in DMEM/F12 (50:50) with 0.2% BSA, 1% vol/vol ITS-X, ascorbic acid, glutamine, MTG, Dex (40 ng/ml), and OSM (5 ng/ml) for 2 days.

Generation of Mature Zone 1 and Zone 3 Hepatocyte-Like Cells from hPSCs-Derived Hepatoblast in 3D Aggregates

Day 27 hepatoblasts were cultured in DMEM/F12 (50:50) with 0.2% BSA, 1% vol/vol ITS-X, ascorbic acid, glutamine, MTG, Dex (40 ng/ml), and OSM (5 ng/ml) for 6 days in monolayer. Day 33 hepatoblasts were dissociated using collagenase type 1 enzyme to make a small cluster of hepatoblasts. The dissociated small clusters were maintained in a low cluster culture dish and cultured in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), and CHIR99021 (1 μM) for 6 days to promote the maturation in 3D aggregates. To induce the differentiation of zone 1-like hepatocytes, 3D aggregates were cultured in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), T3 (Triiodothyronine, 40 nM; Sigma), and XAV939 (2 μM) for 18 days, whereas, to induce the differentiation of zone 3-like hepatocyte, 3D aggregates were cultured in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), T3 (Triiodothyronine, 40 nM; Sigma), and CHIR99021 (1 μM) for 18 days. The medium was changed every two or three days. The differentiation was maintained in an ambient O2 incubator.

Generation of Mature Zone 1 and Zone 3 Hepatocyte-Like Cells from hPSCs-Derived Hepatoblasts in Monolayer Culture

To induce the differentiation of zone 1- and zone 3-like hepatocyte from day 27 hepatoblasts in monolayer culture condition, the hepatoblasts were directly cultured in DMEM/F12 (50:50) based maturation medium in the presence of small molecule that either activate or inhibit of Wnt signalling pathway. For the differentiation of zone 1-like hepatocytes, day 27 hepatoblasts were cultured in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), T3 (Triiodothyronine, 40 nM; Sigma), SB431542 (6 μM), Notch inhibitor: L-685,458 (5 μM) or DAPT (25 μM) and XAV939 (2 μM) for 24 days, whereas, to induce the differentiation of zone 3-like hepatocyte, day 27 hepatoblasts were cultured in DMEM/F12 (50:50) medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), T3 (Triiodothyronine, 40 nM; Sigma), SB431542 (6 μM), Notch inhibitor: L-685,458 (5 μM) or DAPT (25 μM), and CHIR99021 (1 μM) for 24 days. The medium was changed every two or three days. The differentiation was maintained in an ambient O2 incubator.

### Cholangiocytes

Cholangiocyte Differentiation in Monolayer

OP9 cells were maintained as described previously. 30 Gray irradiated OP9 cells were plated on 2.5% Matrigel coated wells (12 well plates) at a concentration of 200,000 cells per well in alpha-modified minimum essential media (a-MEM) supplemented with glutamine (2 mM) and 20% fetal bovine serum. To induce the cholangiocytes differentiation, day 27 hepatoblasts were dissociated using collagenase type I enzyme and then plated onto the irradiated OP9 cells. The plated cells were cultured in H21 DMEM/Ham's F12 (3:1) media supplemented with 0.1% BSA, 1% vol/vol B27 supplement, ascorbic acid, glutamine, MTG, HGF (20 ng/ml), and epidermal growth factor (EGF, 50 ng/ml) for 4 days. To induced CFTR expression in cholangiocyte like cells, following HGF and EGF treatment, the medium was switched into DMEM/F12 medium with 0.1% BSA, 1% vol/vol B27 supplement, ascorbic acid, glutamine, MTG, Retinoic Acid (RA, 1 μM: Sigma: treatment range from 500 nM to 2 μM) for another 6 days. Similar effects were observed when Retinol, AM580 (an RA receptor alpha agonist) or AC55649 (a RA receptor beta agonist) was used. To promote the maturation of cholangiocytes that express primary cilia and 4D9, the cells were cultured with DMEM/F12 medium with 0.1% BSA, 1% vol/vol B27 supplement, ascorbic acid, glutamine, MTG, Noggin (50 ng/ml), ROCK inhibitor Y-27632 (5 μM) and Forskolin (FSK, 5 μM) for 12 days. The medium for all steps of cholangiocytes differentiation were changed every two days. The cells were maintained in an ambient O2 incubator.

Generation of 3D Cholangiocyte Organoids

Day 49 cholangiocyte obtained following the differentiation in monolayer were dissociated with collagenase type I enzyme and then small clumps of cholangiocyte cells were plated on low attachment cluster dishes and cultured with the same medium that was used for monolayer differentiation. The 3D cholangiocyte organoids spontaneously formed cyst like structures within 6 days. The cells were maintained in an ambient O2 incubator.

Generation of Stellate Cells

Stellate cells were obtained from cholangiocyte organoids by culturing the cholangiocyte organoids in DMEM/F12 medium supplemented with 0.2% BSA, 1% vol/vol ITS-X, ascorbic acid, 1% vol/vol chemically defined lipid mix medium (Thermo Fisher Scientific), 0.5% vol/vol B27, glutamine, MTG, Dex (40 ng/ml), CHIR99021 (1 μM), SB431542 (6 μM) and FGF19 (50 ng/ml) for 6 days. After day 6, CHIR99021, SB431542 and FGF19 were removed from the medium, which resulted in the maturation of the cholangiocyte organoids into stellate cells.

FLIPR Membrane Potential Assay

The FLIPR membrane potential assay was conducted following the protocol previously described (Ahmadi et al., 2017, “Phenotypic profiling of CFTR modulators in patient-derived respiratory epithelia,” Genomic Med., 2:12). This assay can be used to measure the apical chloride conductance, which represents CFTR protein functional activity in the cells. In brief, day 27 hepatoblasts were dissociated and plated on 96-well plates with clear bottoms (Corning). Following 4 days of culturing with HGF and EGF, the cells were treated with different concentration of Retinoic Acid including 2 μl of DMSO as a control for 6 days. Prior to the assay, cells were incubated in 200 μL NMDG-gluconate buffer (150 mM NMDG-Gluconate, 3 mM KCl, 10 mM HEPES, pH 7.35, osmolarity 300 mOsm) containing 0.5 mg/mL FLIPR membrane potential dye (Molecular Devices) for 40 mins at 37° C. Following the dye loading procedure, the cells were transferred to the SpectraMax i3X plate reader (Molecular Devices) and their fluorescence was measured using an excitation of 530 nm and an emission of 560 nm with the well-scanning mode on. Baseline fluorescence was measured for 24 minutes (6 minutes/read), followed by the stimulation of CFTR-mediate chloride flux with Forskolin (FSK, 10 μM). After recording membrane potential change for 24 minutes, CFTR function was inhibited with 10 μM CFTRᵢₙₕ-172 for 18 minutes. The raw data was exported and analyzed using the platform established in the laboratory of Christine Bear (The Hospital for Sick Children, Toronto, Canada).

### Example 2B

### Experimental Results for Generating Liver Cells

Hepatocytes and Zonation of Hepatocytes

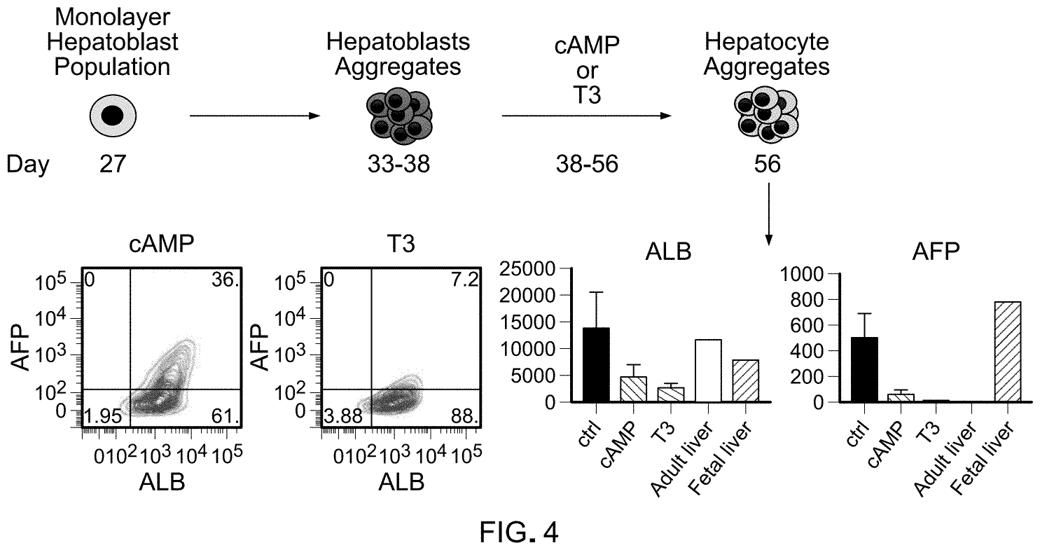

FIG. 4 shows that thyroid hormone (T3) promotes maturation of hPSC-derived hepatocytes. One of the hallmarks of hepatocyte maturation is the downregulation of the fetal gene encoding alpha fetoprotein (AFP) in conjunction with the upregulation of expression of genes associated with adult hepatocyte function. Although a number of different protocols have been described in the literature that claim to promote the development of mature hepatocytes, the resulting cells still express relatively high levels of AFP, suggesting that fetal characteristics are retained. Given that the levels of T3 thyroid hormone increase dramatically after birth and it is known to play a pivotal role in development, growth and function of many tissues, T3 was added to the hPSC-derived hepatocyte cultures to determine if T3 would promote maturation of the hPSC-derived hepatocytes. For these studies, T3 was added to the cultures during the maturation step, from days 38 to 56. During this stage, the cells are cultured as aggregate in the presence of 40 ng/ml dexamethasone, as previously described. The effect of T3 was compared to that of cAMP, as the addition of cAMP has previously been shown to promote maturation of hPSC-derived hepatocytes. As shown in FIG. 4, the addition of T3 resulted in a dramatic decrease in the levels of AFP expression, as demonstrated by flow cytometry and qRT-PCR analyses. In many instances, the levels observed were equivalent to those found in the adult liver and significantly lower than the levels observed in cells treated with cAMP. This is the first demonstration that it is possible to generate hPSC-derived mature hepatocytes that express such low levels of AFP.

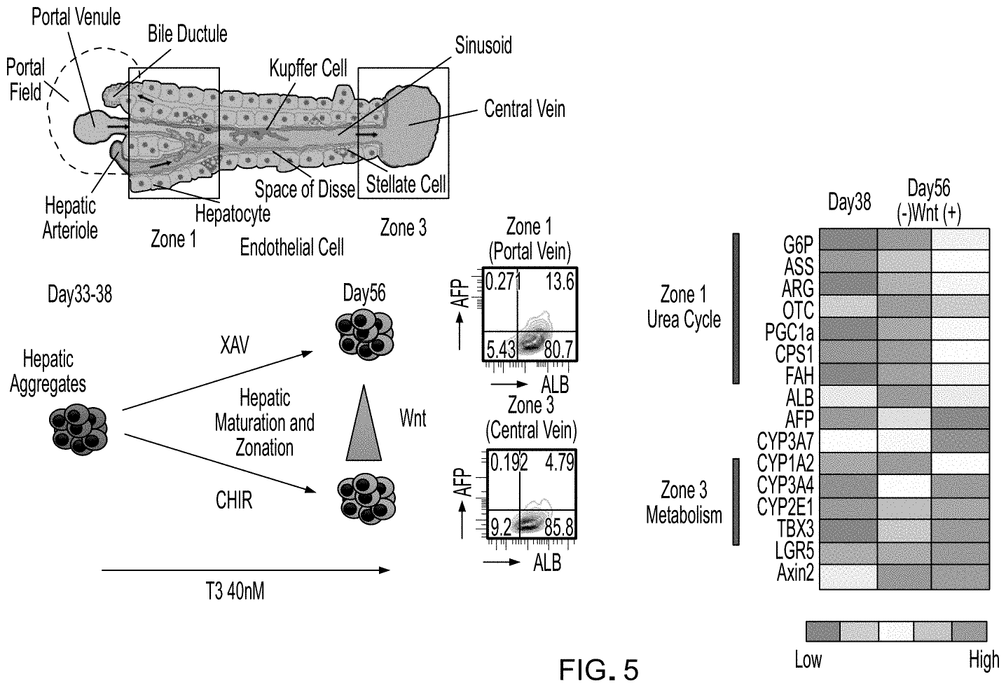

FIG. 5 shows that the Wnt signaling pathway regulates zonation of hPSC-derived hepatocytes. The adult human liver contains distinct populations of hepatocytes that are localized to different regions, or zones, and carry out different functions. To model human hepatocyte development and function from hPSCs, it is essential to generate these different subtypes of cells. Since previous studies in the mouse have shown that Wnt signaling plays a role in development of the different zonated hepatocyte populations, this pathway was manipulated in the culture through the addition of small molecule Wnt agonist, CHIR, or the antagonist, XAV, to the cultures between days 38 and 56 (maturation step). T3 was included in these cultures to promote maturation. As shown in FIG. 5, inhibition of Wnt promotes the development of cells that expressed genes associated with Zone 1 hepatocytes, which are localized in the portal vein regions. These cells upregulate genes associated with fatty acid oxidation, urea production, gluconeogenesis and cholesterol synthesis including ASS, CPS1, ARG1, OTC, PCK1, G6P, and HMGCS2. hPSC-derived hepatocytes generated in the presence of Wnt signaling expressed genes associated with Zone 3 hepatocytes, which are found near the central vein. These cells express genes that encode the P450 enzymes including CYP3A4 and 2D6.

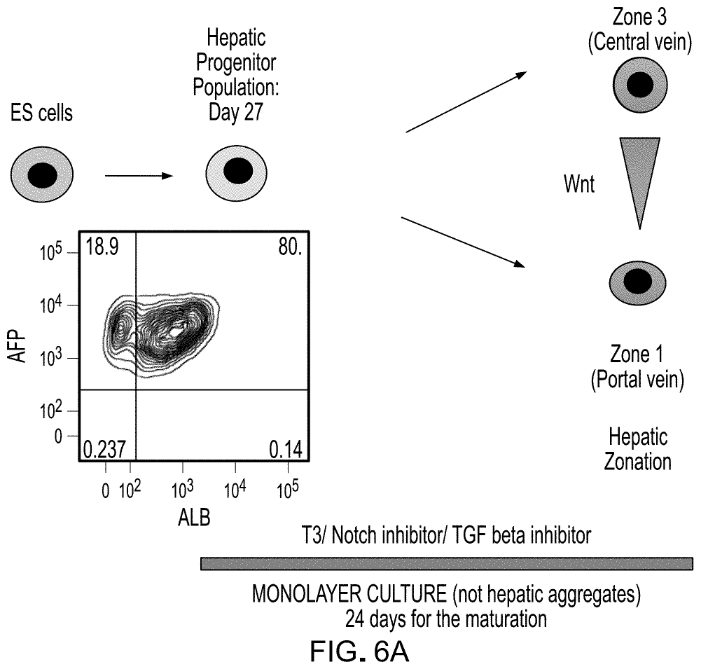

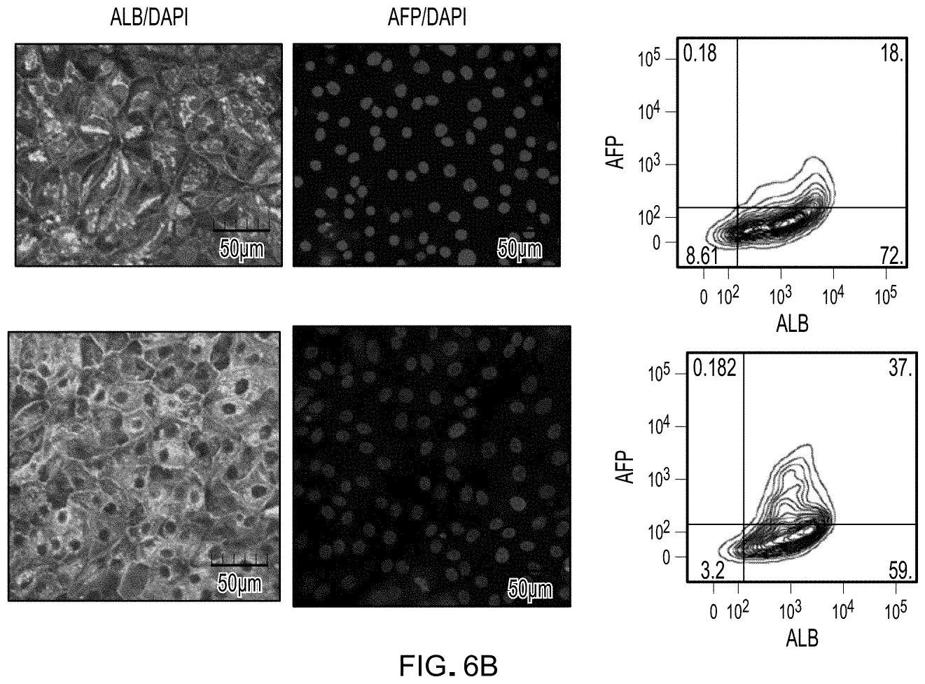

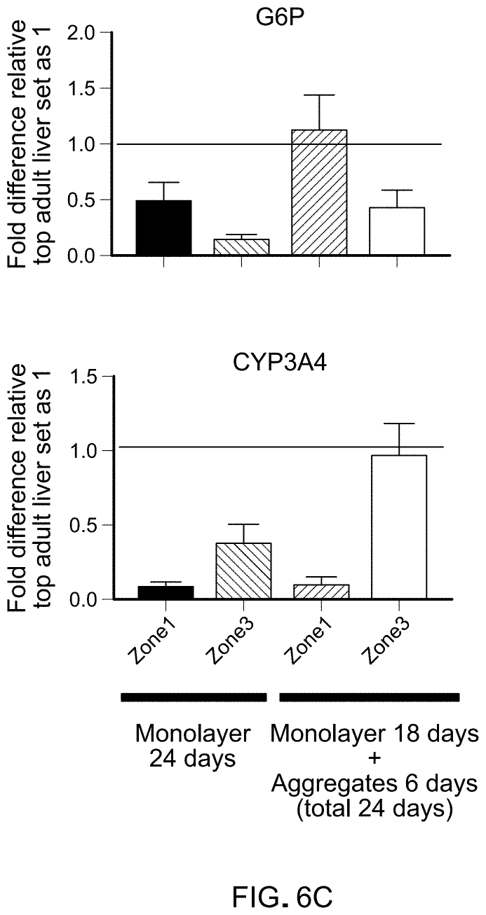

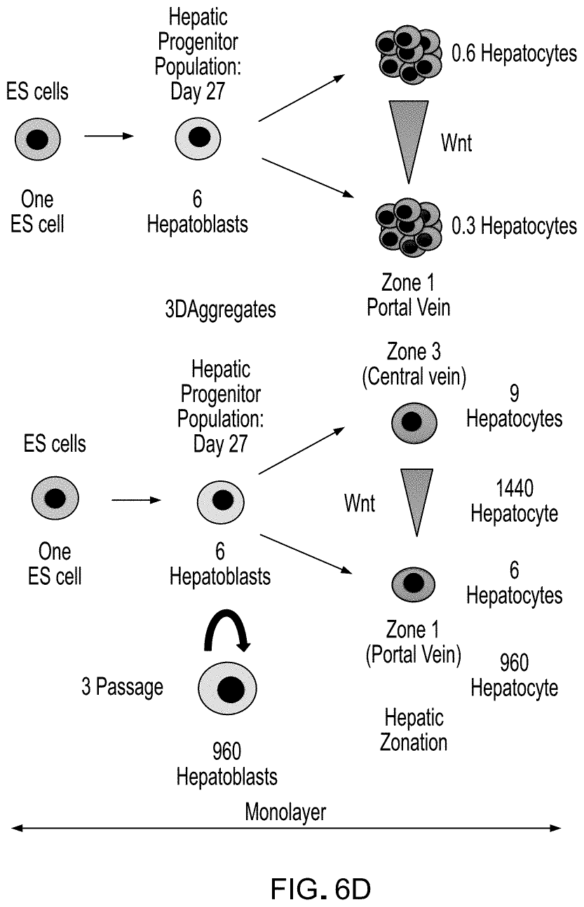

FIG. 6A is a schematic showing that, to promote the hepatic maturation in monolayer culture condition, Notch inhibitor, a TGF beta inhibitor, T3 and modulation of the Wnt signaling pathway was manipulated in the maturation protocol described herein, which caused cells to differentiate into zone 1- and zone 3-like cells. Notch signaling was inhibited by the addition of 0.5 μm-1.0 μM GSI or 25 μM DAPT, and TGF beta signaling was inhibited by the addition of 6 μM SB43152.

FIG. 6B shows that, following 24 days of culturing day 27 hepatoblasts under monolayer conditions, cells expressed albumin and repressed AFP as confirmed by confocal microscopy and flow cytometry. The upper panel displays the characteristics of cells cultured under Zone3 conditions (T3/Wnt agonist/TGFbeta inhibitor/Notch inhibition), whereas the lower panel shows the cells cultured in Zone1 conditions (T3/Wnt agonist/TGFbeta inhibition/Notch inhibition).

FIG. 6C is a graph of qPCR analysis showing that a gluconeogenesis gene, G6P, was upregulated in Zone 1-like cells cultured with Wnt pathway inhibitors, whereas CYP3A4, which is involved in drug metabolism, is upregulated in Zone 3-like cells in the presence of a Wnt pathway agonist. These findings demonstrate that hepatic maturation and zonal manipulation was achieved in monolayer culture condition as well as in 3D aggregates.

FIG. 6D is a schematic showing the estimated number of differentiated Zone 1- and Zone 3-like hepatocytes from one ES cells following the expansion protocol described herein. The methods described herein are able to produce six hepatoblasts from one ES cell after 27 days (top). Following the formation of aggregates and promotion of maturation by addition of Wnt agonist/antagonist and thyroid hormone, 0.6 zone 3-like cells and 0.3 zone 1-like cells can be differentiated from one ES cells (top). Maturation in a monolayer culture with the inhibition of Notch and TGF beta signaling resulted in more than a 10-fold increase in the number of Zone 1/3-like hepatocytes generated compared to the number of cells generated in 3D culture. Following third-passaged expansion of hepatoblasts, over 1000 zone 1/3-like cells can be produced from one human ES cells.

Cholangiocytes

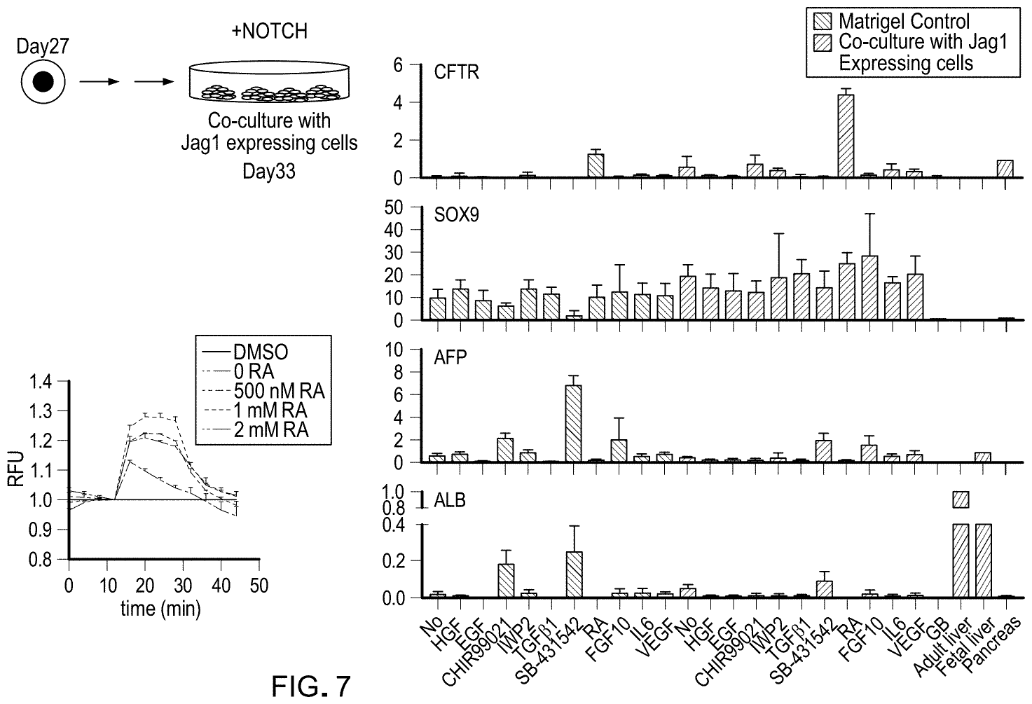

FIG. 7 shows that retinoic acid signaling promotes the generation of CFTR expressing cholangiocytes (bile duct cells) in monolayer cultures. It has previously been reported that it is possible to generate cholangiocytes that express a number of markers that are indicative of mature cells including the cystic fibrosis transmembrane conductance regulator (CFTR) gene, in which mutations cause cystic fibrosis. The development of mature cholangiocytes was dependent on growth of the cells as cysts in 3D semi-solid cultures consisting of Matrigel and collagen. While this approach yielded relatively mature cholangiocytes, the culture system was not amenable to cell expansion or high throughput screening (e.g., for drugs against cystic fibrosis and other biliary diseases). To improve the differentiation efficiency in monolayer cultures, a panel of cytokines and small molecules that are known to play a role in bile duct development were screened to identify those that would promote the upregulation of CFTR expression as an indication of maturation. Since Notch signaling is required for the production of cholangiocytes, hepatoblasts were cultured on either OP9-Jagl cells or Matrigel for 6 days as previously described. From this screen, it was found that retinoic acid (RA) signaling significantly induced CFTR expression in the population co-cultured with OP9-Jagl. To further investigate the role of RA signaling, the effects of specific RA receptor agonists as well as a pan antagonist also were tested. BMS493, a RA receptor antagonist, inhibited the induction of CFTR expression, whereas addition of the RA receptor alpha agonists (AM580), RA receptor beta agonist (AC55649) and RNA receptor gamma agonist (CD437) all induced CFTR expression. These findings show that RA signaling is important for the generation of CFTR-expressing cholangiocytes in the monolayer culture, and cells treated with RA show a functional CFTR response in the FLIPR assay.

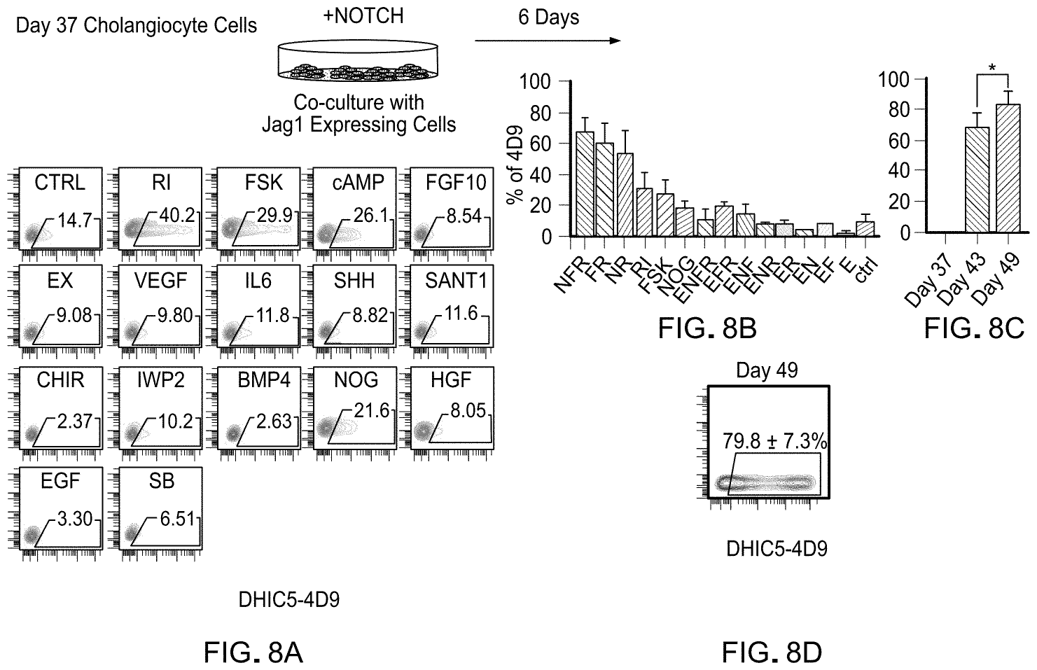

FIG. 8 shows that identification of signaling pathways that promote the development of ciliated cholangiocytes, which are functional bile duct cells, in monolayer cultures. One of the primary determinants of cholangiocyte maturation and function is the development of primary cilia. These cilia extend from the apical plasma membrane into the lumen of the bile duct and function as mechanosensors to deliver signaling initiated by fluid flow in the duct to the cholangiocytes. At a molecular level, cilia development correlates with the upregulation of expression of genes including PDK1, PDK2 and TRPV 4. Although RA signaling induced the expression of CFTR, it did not promote the development of cilia in the cholangiocytes. To identify pathways that promote further maturation of the hPSC-derived cholangiocytes, a screening approach was used based on flow cytometric identification of cells that express the epitope recognized by the antibody DHCS-4D9, which stains mature bile ductal cells (cholangiocytes) in the adult liver. It was hypothesized that maturation to the stage of DHCS-4D9 positivity would correlate with cilia formation. For this screen, different combinations of agonists and antagonist to the following signaling pathways were added to the cultures for 6 days: cAMP, Wnt, Hedgehog, EGF, BMP, HGF, TGF beta, FGF10, IL6, VEGF and Extendin4. Following this maturation step, the cells were harvested and analyzed by flow cytometry for reactivity with DHC5-4D9. As shown in FIG. 8, ROCK inhibitor (R), cAMP signaling (forskolin, F), and inhibition of the BMP pathway (N) all promoted the development of DHC5-4D9 cells. The combination of the three manipulations (NFR) consistently gave rise to the largest proportion of DHC5-4D9+ cholangiocytes, up to 80% of the population.

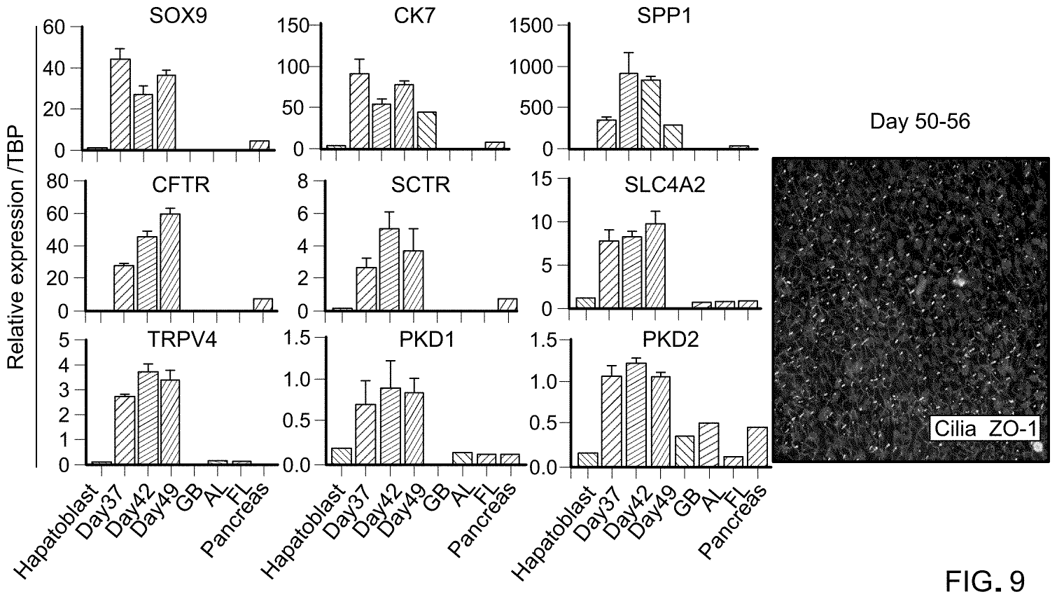

FIG. 9 shows the characterization of NFR-induced cholangiocytes. QRT-PCR-based expression analyses revealed that the cholangiocytes induced with NFR in the monolayer format expressed many genes associated with mature cholangiocytes function, including those involved in cilia formation such as TRPV4, PDK1 and PDK2 (FIG. 9). Additionally, a large majority of the cells contained primary cilia (H9: 77.3±11.0%; 2 cell lines of iPS-derived F508del CF patient lines: 76.1±10.9%, 77.5±4.9%). The cells generated with this protocol show a robust CFTR response in the high throughput FLIPR assay, indicating that they will be appropriate for screening for new CF drugs.

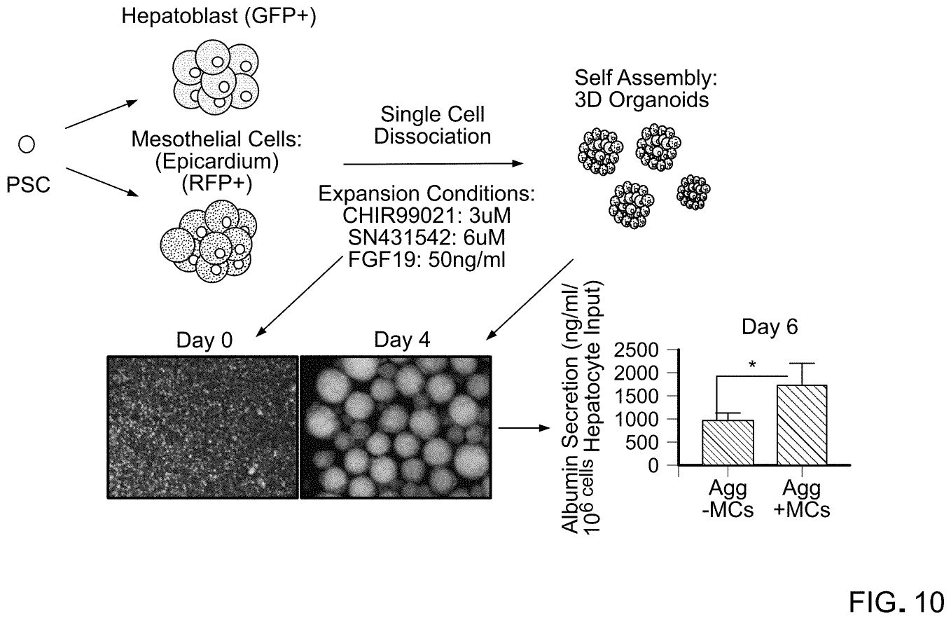

FIG. 10 shows that hPSC-derived mesothelial cells support hepatoblast function in vitro. The adult liver is surrounded by a population of mesothelial cells (MCs) that forms an epithelium around the organ. While the function of this cell population is not fully understood, studies in model organisms suggest that they interact with the hepatocytes and undergo an epithelial-to-mesenchymal transition (EMT) and contribute to the stellate cell population within the liver. To model this interaction in vitro, a mesothelial population was generated from hPSCs using a modification of a published protocol designed for the development of epicardial cells of the heart. The epicardium of the heart and the mesothelium that surrounds the liver share many characteristics including the expression of WT1, RALDH2 and TBX18. To be able to track the MCs, they were generated from a hPSC line that constitutively expresses RFP. Single cell suspensions of day 20-25 mesothelial cells were mixed with day 27 hepatoblasts generated from a hPSC line that constitutively expresses GFP. The cells were mixed at a hepatoblast/MC ratio of 4:1, and the developing aggregates were cultured in the expansion conditions described herein. The cells formed aggregates, referred to as organoids, within 4 days of culture and appeared to segregate to distinct regions within the structure, with the RFP+ mesothelial cells forming a distinct layer around the GFP+ hepatoblasts. This segregation appears to recapitulate the positioning of these cell types in the developing liver. Analyses of day 6 aggregates revealed that those cultured in the presence of the MCs secreted significantly more albumin than those cultured without these cells. Following 6 days in culture, total numbers of hepatoblasts were not significantly different between organoids cultured with and without MCs. Culture in the presence of MCs over a 3-4 week period during the maturation to Zone 1 and Zone 3 fates promoted the survival of the hepatoblasts within the aggregates; those with MCs contained 2-3 fold more cells than those without. These observations suggest that culture with mesothelial cells may provide a novel approach for maintaining hepatoblasts function in vitro.

### Example 3A

### Experimental Materials and Methods for Therapeutic Applications

Liver injury in mice was induced by administration of GSV in 6-8 weeks TK NOG mice. Day 50-56 differentiated cholangiocytes in monolayer conditions were dissociated by TrypLE to make a single cell suspension. Under proper anesthesia, a skin incision was made in the left abdomen under the rib. The abdomen was entered through the same incision. The spleen was gently mobilized from the incision. One million cholangiocytes in 50 μl PBS were injected at the lower pole of the mobilized spleen. After confirmation of hemostasis at the injection site, the skin incision was closed. Animals were then euthanized six weeks following the transplantation, and the liver was removed and fixed for immunohistochemical study. For immunostaining of human CK19 and mitochondria, paraffin-embedded sections were dewaxed and subjected to heat-induced epitope retrieval. Following standard immunostaining methods, transplanted iPSCs derived cholangiocyte was confirmed based on the presence of human mitochondria and CK19-positive cells.

Liver organoids were made self-assembling with GFP-positive hepatoblasts and RFP-positive mesothelial cells differentiated from hPSCs. Keeping aggregation in culture medium for six days, liver organoids composed of 6 million hepatoblast were embedded into 2.4 mg/ml collagen type 1 gel with 1-2 million human umbilical cord endothelial cells (HUVEC). After solidification of the collagen gel, the gel containing the liver organoids in the presence or absence of HUVEC was removed from the culture plate and transplanted under the back skin of NOG mice. Six weeks following the transplantation, the transplanted mouse was euthanized, and the transplanted tissue was removed for immunohistochemical analysis. Before euthanization, blood samples were collected to measure human serum albumin.

Liver organoids containing 6 million hepatoblasts and 1.5 million mesothelial like cells were embedded in 2.4 mg/ml collagen type 1 gel in the presence of 1-2 million HUVEC. Under proper anesthesia, the laparotomy was made with a central abdominal incision. After the middle and lateral segment of mouse liver was removed with ligation, one or two solidified collagen gel with liver organoids and HUVEC were implanted on the surface of proximal mesentery nearby the liver. Implanted gel on the intestinal mesentery was covered with SURGICEL to prevent movement. As a control experiment, collagen gel with liver organoids and HUVEC were implanted at the same site without partial hepatectomy. 4 weeks after the transplantation, blood samples were collected to measure the human serum albumin.

### Example 3B

### Experimental Results for Therapeutic Applications

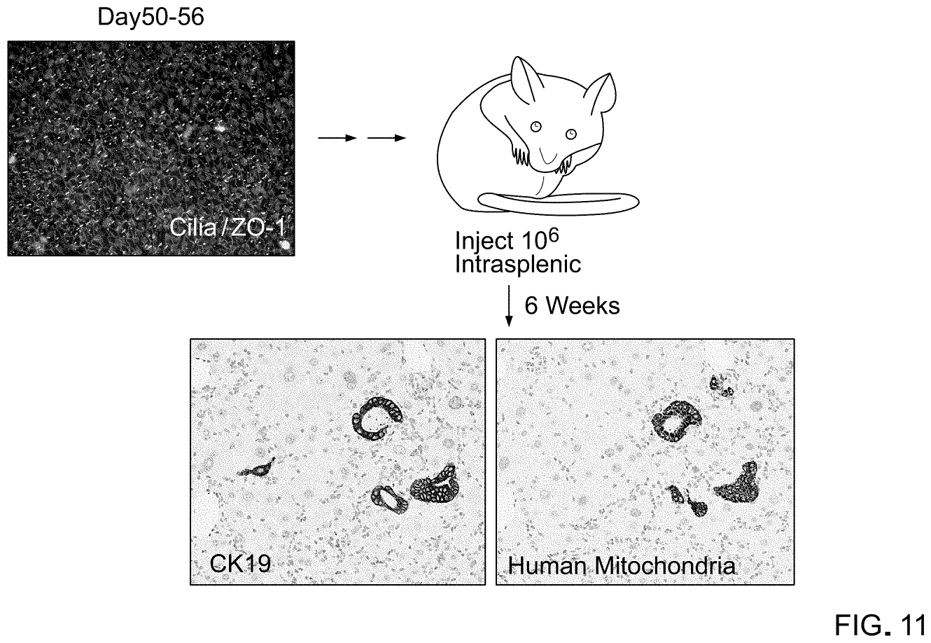

FIG. 11 shows engraftment of hPSC-derived cholangiocytes. To determine if the NFR-induced cholangiocytes can function in vivo, 1×10E6 mature, day 62 cells were transplanted into ganciclovir-treated TK-NOG mice. Treatment of these engineered mice with ganciclovir kills the host mouse hepatocytes and enables engraftment of human cells. Mice were sacrificed 6 weeks following transplantation and their livers were analyzed for the presence of human cholangiocytes. In two independent experiments, ductal structures consisting of human cytokeratin 19 (CK19) positive cells were detected in the livers of all recipients. These findings are the first to demonstrate engraftment of hPSC-derived cholangiocytes into the liver of a mouse.

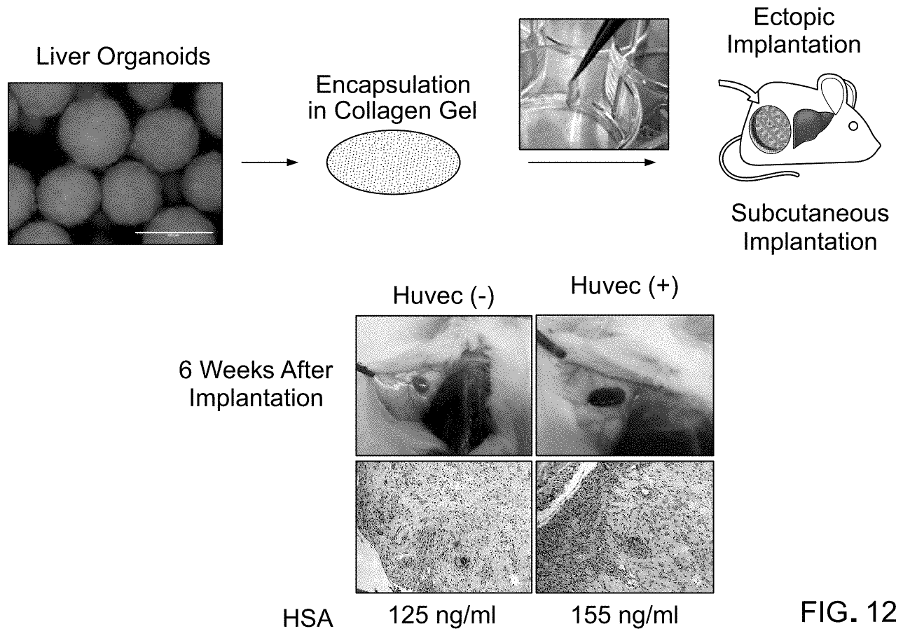

FIG. 12 shows the subcutaneous (ectopic) transplantation and engraftment of hepatic organoids. To determine if the hepatic organoids can function in vivo, day 27 organoid generated with mesothelial cells and hepatoblasts were encapsulated in a collagen gel with or without HUVEC endothelial cells. The gels were transplanted in a subcutaneous site in NSG recipients. Six weeks following transplantation, the mice showed measurable levels of human albumin in their sera (HSA). Grafts could be detected in all transplanted mice, and those whose transplant included HUVEC tended to be larger than those that did not. Histological analyses showed that the graft contained albumin-positive hepatocyte clusters (arrow heads) surrounded by small capillary blood vessels containing red blood cells (arrows). Together, these preliminary findings show that the liver organoids can engraft ectopic sites and function to produce HSA over a 6-week period. Hepatoblast aggregates without MCs generated grafts consisting of fibrotic tissue, with few albumin-positive cells, suggesting that mesothelium cells supports the development of functional hepatocytes in vivo.

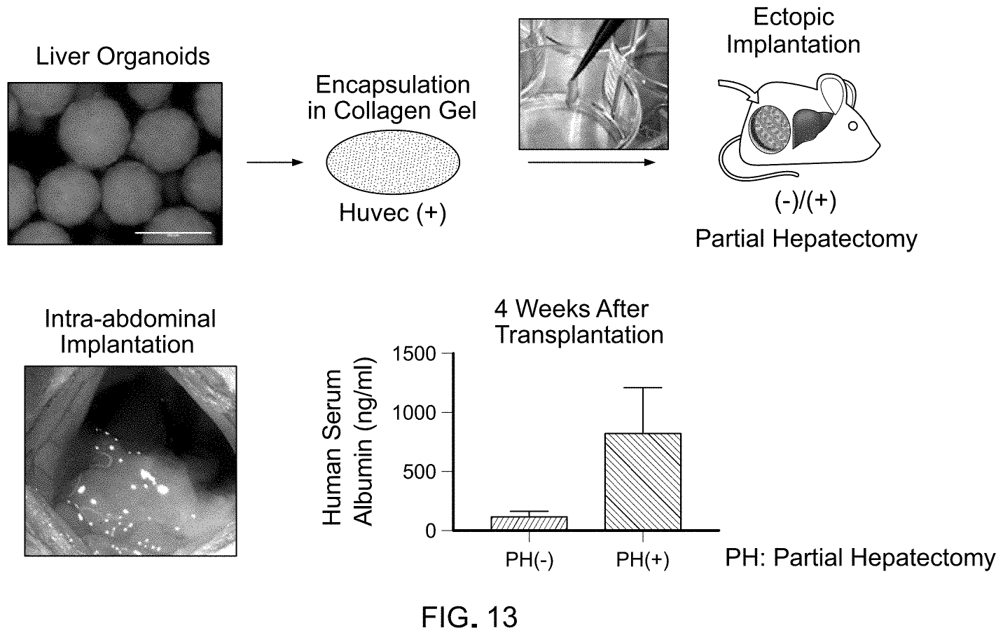

FIG. 13 shows the intra-abdominal (ectopic) transplantation and engraftment of hepatic organoids. In this set of experiments, partial hepatectomy was performed on the recipient NSG mice prior to transplantation of the collagen gel containing organoids and support HUVEC cells. Following surgery, the gel was positioned in the hepatic hilum covering the portal vessels and bile ducts. Four weeks following transplantation, HSA was detected in the sera of all animals (n=3). The levels in those that had undergone partial hepatectomy were much higher than in those without the surgery, suggesting that increased demand for liver function provides a stimulus to improve engraftment and/or function of the ectopic tissue.

### Example 4

### Cryopreservation and Expansion of hPSCs-Derived Hepatoblasts

The protocol for cryopreservation of hepatoblasts: Day 27 hepatoblasts were expanded with a treatment of FGF19/SB43152/CHIR99021 (“expansion cocktail”) for 6-8 days. Medium was changed every two days. Expanded hepatoblasts were dissociated with TrypLE for 5 minutes and harvested as single cell hepatoblasts. The cryopreservation of hepatoblasts was carried out with the conventional cryopreservation methods in the presence of 10% DMSO, 40% FSC and 50% DMEM/F12 at a density of 0.5-1.0 million cells per frozen vial.

Thawing cryopreserved hepatoblast: after thawing the cryopreserved hepatoblasts in a water bath for 5 minutes, the cells were washed with DMEM/F12 one time and resuspended into fresh DEME/F12 containing the expansion cocktail. The recovered hepatoblasts were plated at a density of 1.0×10e5 cells per a well in 12 well culture plate dish in DEME/F12 containing the expansion cocktail and 10 μM Rho-kinase (Rock) inhibitor. Rock inhibitor was no longer included after the first media change at 48 hours, and the media was changed every 48 hours thereafter until the hepatoblast population reached confluency.

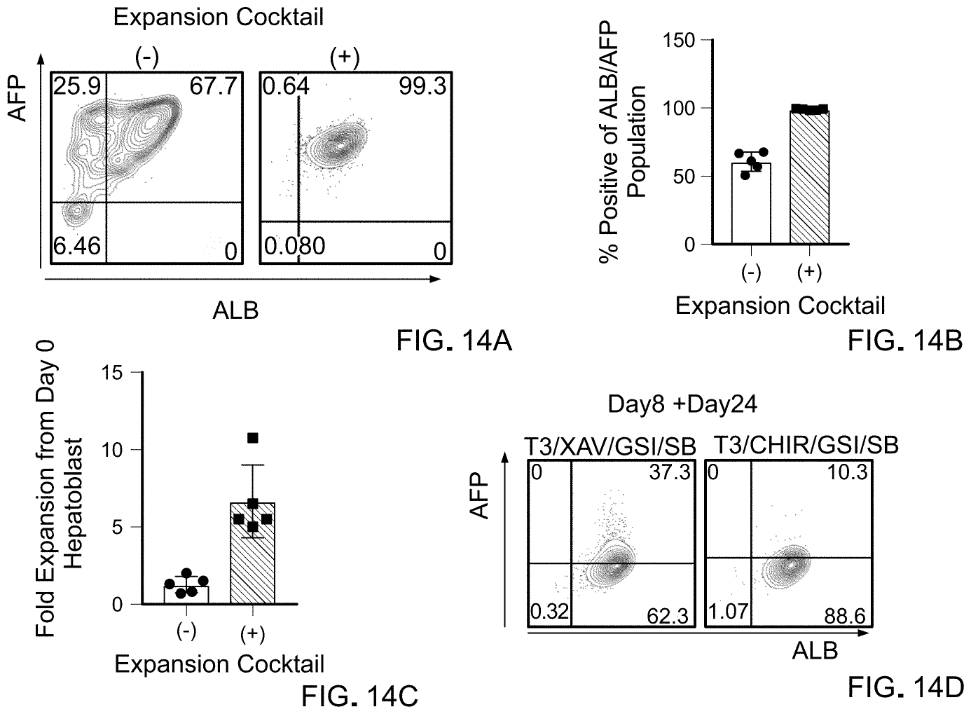

1.0×10e5 hepatoblasts in each well of a 12-well culture dish plate (3.5 cm2) were plated and cultured in the expansion medium. Medium was changed every two days until the hepatoblast population achieved confluency at day 8 of culture. At this stage, the cell number increased on average 6.65±2.35-fold and more than 98% of the cells in the population expressed both ALB and AFP (FIG. 14A, 14B, 14C). Populations that were not expanded in the expansion cocktail prior to cryopreservation did not expand following the thaw when cultured in expansion cocktail. The population that did persist following 8 days of culture contained a substantial proportion of ALB− cells. These findings show that expansion of the hepatoblast population prior to cryopreservation can allow for an improved recovery of a functional cell population that can be further expanded and differentiated to mature zonated hepatocytes.

When thawed and cultured (8 days) hepatoblasts were subjected to Zone1 (T3, XAV, GSI and SB) or Zone3 (T3, CHIR, GSI and SB) maturation stimuli, they differentiated to give rise to distinct populations that contained few AFP+ cells (FIG. 14D) and displayed the expected Zone 1 and Zone 3 gene expression patterns. In addition to mature zonated hepatocytes, the cryopreserved hepatoblasts were also able to generate functional CFTR+ ciliated cholangiocytes when cultured under the cholangiocyte inducing/maturation conditions (data not shown).

### Example 5

### Ectopic Kidney Subcapsular Transplantation of hPSCs-Derived Zonated Hepatic Aggregates into NSG Mice

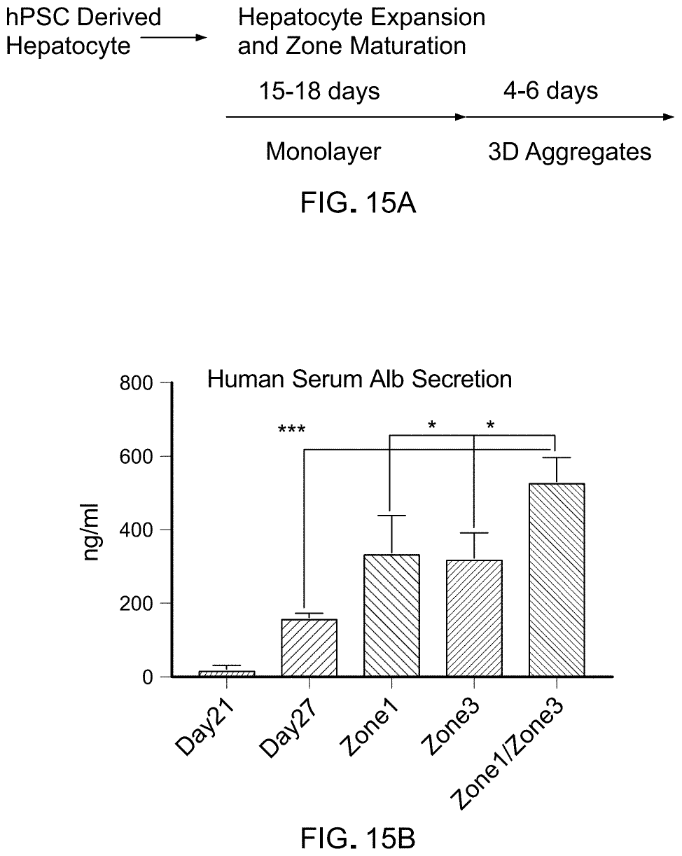

To explore the functional capacity of the differentiated hepatocytes in vivo, aggregates of day 21 or day 27 hepatoblasts or Zone1/Zone3-matured hepatocytes were ectopically transplanted into the kidney subcapsule of NSG mice. Zone1- and Zone3-like hepatocytes were differentiated in monolayer cultures from expanded, non-cryopreserved hepatoblasts. The expanded hepatoblasts were subjected to Zone 1 (T3, XAV, GSI and SB) or Zone 3 (T3, CHIR, GSI and SB) maturation stimuli in the monolayer condition. Following 15-18 days of monolayer culture, 3D aggregates were generated from the monolayer cells and maintained in culture for an additional 4-6 days. Aggregates were also generated from the day 21 and day 27 hepatoblasts. Aggregates from the different populations were transplanted to kidney subcapsular space of NSG mice. Each mouse received the number of aggregates generated from 8-10×10e6 monolayer cells. For the mixed populations, Zone 1 and Zone 3 aggregates were mixed in equal proportions (the equivalent of 4-5×10e6 monolayer cells of each) prior to transplantation (FIG. 15A).

Human serum albumin (HSA) was measured by ELISA in the sera four weeks following the transplantation. Mice transplanted with either Zone1 or Zone3 hepatic aggregates had higher levels of HSA than those transplanted with aggregates generated from Day 21 and Day 27 progenitor cells. Notably, mice that received the mixture of Zone1 and Zone3 aggregates showed the highest levels of HSA (FIG. 15B). These data suggest that both Zone1 and Zone3 hepatocytes cooperate to maintain the hepatic function in vivo.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these also is specifically contemplated and disclosed.

1. A method of expanding hepatoblasts, comprising:
culturing the hepatoblasts in the presence of an activator of the Wnt pathway, a TGF beta inhibitor, and FGF19 or an equivalent thereof.

2. The method of claim 1, wherein the activator of the Wnt pathway is CHIR99021, CHIR98014, BIO, a GSK-3 beta inhibitor, or a natural Wnt agonists such as Wnt3.

3. The method of claim 1, wherein the TGF-beta receptor inhibitor is SB431542, A83-01, or an ALK4 and/or ALK7 inhibitor.

4. The method of claim 1, wherein the FGF19 or an equivalent thereof is NGM282.

5. The method of claim 1, wherein the method is performed under hypoxic conditions.

6. A method of expanding hepatoblasts, comprising:
culturing the hepatoblasts under hypoxic conditions.

7. The method of claim 6, further comprising culturing the hepatoblasts in the presence of an activator of the Wnt pathway, a TGF-beta receptor inhibitor, and FGF19 or an equivalent thereof.

8. A method of expanding hepatoblasts, comprising:
culturing the hepatoblasts under hypoxic conditions in the presence of an activator of the Wnt pathway, a TGF beta inhibitor, FGF19 or an equivalent thereof.

9. The method of claim 1, wherein the number of hepatocytes are expanded about 100-fold to about 400-fold within 3 to 5 passages when cultured under ambient O2 conditions.

10. The method of claim 5, wherein the number of hepatocytes are expanded about 75-fold to about 1000-fold within 3 to 5 passages when cultured under hypoxic conditions.

11. A method of obtaining mature hepatocytes, comprising:
culturing hepatoblasts in the presence of a thyroid hormone or a thyroid hormone receptor agonist.

12. The method of claim 11, wherein the thyroid hormone is triiodothyronine or thyroxine.

13. The method of claim 11, wherein the thyroid hormone receptor agonist is GC-1.

14-18. (canceled)

19. A method of producing Zone 1 hepatocytes, Zone 3 hepatocytes, or cholangiocytes, comprising:
culturing hepatoblasts in the presence of an inhibitor of the Wnt pathway, in the presence of an activator of the Wnt pathway, or in the presence of retinoic acid, retinol or a RA receptor agonist, respectively.

20. The method of claim 19, wherein the inhibitor of the Wnt pathway is XAV939, IWP2, IWP4, or ICRT14.

21. The method of claim 19, wherein the hepatoblasts are cultured in a monolayer or in aggregates.

22-23. (canceled)

24. The method of claim 19, wherein the hepatoblasts are cultured in the presence of a NOTCH inhibitor.

25-32. (canceled)

33. A method of treating a subject having liver disease, comprising transplanting a composition into the subject, wherein the composition comprises
hepatoblasts expanded using the method of claim 1.

34-40. (canceled)

41. A method of cryopreserving liver cells, comprising:
culturing the liver cells in the presence of an activator of the Wnt pathway, a TGF beta inhibitor, and FGF19 or an equivalent thereof for at least 3 days; and
cryopreserving the cultured liver cells.

42. The method of claim 41, further comprising thawing the cryopreserved liver cells and culturing the thawed liver cells in the presence of an activator of the Wnt pathway, a TGF beta inhibitor, and FGF19 or an equivalent thereof.

43. A method of recovering cryopreserved liver cells, comprising:
thawing the cryopreserved liver cells; and
culturing the thawed liver cells in the presence of an activator of the Wnt pathway, a TGF beta inhibitor, and FGF19 or an equivalent thereof.

44. The method of claim 43, further comprising culturing the liver cells in the presence of an activator of the Wnt pathway, a TGF beta inhibitor, and FGF19 or an equivalent thereof for at least 3 days prior to cryopreserving the liver cells.

45. The method of claim 41, wherein cryopreservation comprises freezing the liver cells at −80° C. in media comprising DMSO, FSC and DMEM/F12.

46. The method of claim 41, wherein thawing comprises heating the liver cells to 37° C. for about 5 mins.

47. The method of claim 41, wherein the liver cells are hepatoblasts.

48. (canceled)
